(19) United States
(12) Patent Application Publication
(10) Pub. No.: US 2003/0073744 A1
(43) Pub. Date: Apr. 17, 2003

(54) Agent for enhancing cerebral acetylcholine release

(76) Inventors: Kazuo Nakamura, Mishima-shi (JP); Masatoshi Shirane, Fujisawa-shi (JP); Yushiro Tanaka, Yokohama-shi (JP)

Correspondence Address: HOFFMANN-LA ROCHE INC., PATENT LAW DEPARTMENT, 340 KINGSLAND STREET, NUTLEY, NJ, 07110

(21) Appl. No.: 10/309,434

(22) Filed: Dec. 4, 2002

Related U.S. Application Data
(63) Continuation of application No. 09/557,142, filed on Apr. 25, 2000.

(30) Foreign Application Priority Data
Apr. 27, 1999 (EP) 99108223.1

(51) Int. Cl. A61K 31/195; A61K 31/192

(52) U.S. Cl. 514/563; 514/568

(57) ABSTRACT

The present invention relates to the utilization of N-anisoyl-γ-aminobutyric acid (N-anisoyl-GABA) or p-anisic acid, for the preparation of a pharmaceutical composition that enhances acetylcholine (ACh) release in the brain. In particular, the invention is concerned with the use of N-anisoyl-GABA or p-anisic acid for the treatment of circadian rhythm disorders, sleep disorders, attention deficit disorders and problematic behaviors. The invention also relates to pharmaceutical compositions comprising N-anisoyl-GABA or p-anisic acid as an effective ingredient.

This application is a Continuation of U.S. patent application Ser. No. 09/557,142, filed Apr. 25, 2000.

### BACKGROUND OF THE INVENTION

Acetylcholine (ACh) has been well known to be an important classical neurotransmitter. The major cholinergic pathways exist in the mesopontine reticular nucleus-thalamus, septum-hippocampus and forebrain basal nucleus-neocortex in mammalian brains [Neuroscience, 10, 1185-1201 (1983)]. These cholinergic pathways have been thought to play a functionally pivotal role in the induction and maintenance of rapid-eye movement sleep (REMS) and in the regulation of vigilance and attention, learning and memory, and motivation [Journal of Neuroscience, 10, 2541-2559 (1990); Brain Research Review, 19, 298-318 (1994)]. On the other hand, central cholinergic neuronal activity was decreased in patients with neuronal degeneration (i.e., Alzheimer's disease, Parkinson's disease and progressive supranuclear palsy) [Lancet, 2, No.8000, 1403 (1976); Journal of Neurological Neurosurgical Psychiatry, 51, 540-543 (1988)] and with cerebrovascular diseases [Dementia, 5, 163-167 (1994); Journal of Neural Transmission, 103, 1211-1220 (1996)]. The cholinergic deficit and/or dysfunction has been suggested to be associated with various neuropsychiatric symptoms, such as dementia, sleep disorder, low vigilance, attention deficit and problematic behaviors including delirium and nocturnal wandering.

1-p-Anisoyl-2-pyrrolidinone (aniracetam, European Patent Nos. 5143 and 44088), a cerebral function improver, has been used for the treatment of emotional disturbances (anxiety/irritation and depressive moods) appearing as sequelae after cerebral infarction [Igaku No Ayumi, 156, No.2, 143-187 (1991); Geriatric Medicine, 36, 1513-1520 (1998)]. Though it has been reported that the drug activates the central cholinergic systems, it remains unclear which substance(s), including its metabolites, is actually responsible for the cholinergic activation by orally administered 1-p-anisoyl-2-pyrrolidinone [Drug Investigation, 5, 1-108 (1993)]. Furthermore, the biological activities of N-anisoyl-γ-aminobutyric acid (N-anisoyl-GABA) and p-anisic acid, which are the major in vivo metabolites of 1-p-anisoyl-2-pyrrolidinone, are not well clarified.

In the meantime, the treatment for the diseases described above is begun by increasing central ACh levels with ACh esterase inhibitors. Because ACh esterase is widely distributed not only in the brain but also in the peripheral tissues, the peripheral side effects caused by the poor penetration of ACh esterase inhibitors through the blood-brain barrier may produce serious problems for these enzyme inhibitors as a medicine. Hence, the problem to be solved by the present invention was to provide promising compounds, which are able to safely increase central ACh levels.

### SUMMARY OF THE INVENTION

The present invention relates to the use of N-anisoyl-GABA or p-anisic acid, preferably in the form of a pharmaceutical composition, for enhancing ACh release in the brain of mammals, preferably humans. These compounds and pharmaceutical compositions comprising N-anisoyl-GABA or p-anisic acid are particularly useful for the treatment of circadian rhythm disorders, sleep disorders, attention deficit disorders and problematic behaviors. It has now been found that surprisingly N-anisoyl-GABA and p-anisic acid each enhanced cerebral ACh release in the brain. Since the pharmacological potency of N-anisoyl-GABA is slightly stronger it is the preferred compound in connection with the present invention.

### BRIEF DESCRIPTION OF THE DRAWINGS

FIG. 1: Effects of aniracetam (A), 2-pyrrolidinone, p-anisic acid and N-anisoyl-GABA (B) on ACh release in the reticular nucleus of the thalamus of freely moving stroke-prone spontaneously hypertensive rats (SHRSP). Compounds were perfused for 20 minutes (solid bar). Data represent means±S.E.M. Basal ACh release was 0.35±0.03 pmol/20 min (n=24). * P<0.05 compared with vehicle control.

FIG. 2: Effects of aniracetam (A), 2-pyrrolidinone, p-anisic acid and N-anisoyl-GABA (B) on ACh release in the dorsal hippocampus of freely moving SHRSP. Compounds were perfused for 20 minutes (solid bar). Data represent means±S.E.M. Basal ACh release was 0.63±0.04 pmol/20 min (n=15). * P<0.05, ** P<0.01 compared with aniracetam.

FIG. 3: Effects of aniracetam (A), 2-pyrrolidinone, p-anisic acid (B) and N-anisoyl-GABA (C) on ACh release in the prefrontal cortex of freely moving SHRSP. Compounds were perfused for 20 minutes (solid bar). Data represent means±S.E.M. Basal ACh release was 0.58±0.03 pmol/20 min (n=24). * P<0.05, ** P<0.01 compared with aniracetam.

FIG. 4: Circadian motor activity rhythm and mealtime-associated anticipatory activity in young and aged rats. After fasting for 24 hours (Day 0), feeding was restricted to only 1 hour from 13:30 for 6 consecutive days (Days 1 to 6). Food was withheld again on Day 7. Data show means±S.E.M of motor activity measured every 1 hour, obtained from 7 rats per group. Dotted column indicates the feeding period. Δ:=Day −1; ◯:=Day 6; ●:=Day 7

FIG. 5: Effects of aniracetam on circadian motor and mealtime-associated anticipatory activity rhythms in aged rats. Aniracetam was given orally to rats immediately after the termination of feeding once daily for 7 consecutive days (Days 1 to 7). Data show means±S.E.M. obtained from 5-6 rats per group. Dotted column indicates the feeding period. ◯:=Day 6; ●:=Day 7

FIG. 6: Diurnal and nocturnal changes of REMS, non-REMS (NREMS) and brain temperature in SHRSP and age-matched Wistar Kyoto rats (WKY). Data show means±S.E.M. of each variable measured every 1 hour.

FIG. 7: Effects of repeated administration of aniracetam on REMS, NREMS and brain temperature in SHRSP. Aniracetam was given orally to animals twice daily (9:00 and 20:00) for 5 consecutive days, and the data after the 9th and 10th dosages are shown.

### DETAILED DESCRIPTION OF THE INVENTION

The invention relates to pharmaceutical compositions for enhancing cerebral acetylcholine release comprising N-anisoyl-GABA or p-anisic acid as an effective ingredient for the treatment of the above disorders.

The invention also relates to methods of using N-anisoyl-GABA or p-anisic acid for enhancing ACh release in the brain, in particular for the treatment of circadian rhythm disorders, sleep disorders, attention deficit disorders and problematic behaviors.

The present invention is explained in detail below. The description and the examples provided below will help to understand the invention, but should not be seen as limiting the invention in any way. The results obtained in the examples are documented by the accompanying figures.

Both N-anisoyl-GABA and p-anisic acid are known chemical compounds. N-anisoyl-GABA can be synthesized by the methods described in the Spanish Patent Publication No. 84-538772. p-Anisic acid can be produced by the methods described in the Journal of American Chemical Society 78, 907-909 (1956) and also purchased from Sigma Chem. Co. (St. Louis, USA), Lancaster Synthesis Ltd. (Lancashire, UK), Wako Pure Chem. Ind. Ltd. (Osaka, Japan), and so on.

N-anisoyl-GABA and p-anisic acid can each be used in the form of a pharmaceutically acceptable preparation. This preparation can be formed into tablets, coated tablets, confections, hard gelatin capsules, soft gelatin capsules, as well as into solution, emulsion or suspension. The resulting formulation can be administered orally. In addition, this preparation can be formed into suppositories for intrarectal administration, or into injectable form that can be administered parenterally.

When peroral solid preparations, such as tablets, coated tablets, confections, or hard gelatin capsules are prepared, N-anisoyl-GABA or p-anisic acid can each be formulated together with pharmaceutically inert inorganic or organic carriers, such as lactose, maize or corn starch and their derivatives, talc, stearic acid and its bases or salts, and so on.

When soft or hard gelatin capsule products are formed, carriers, for example, vegetable oils, waxes, fats, oils, gels, semi-solid or liquid polyols, and so on, can be used appropriately.

When liquid and syrup products are formed, carriers, for example, water, polyols, saccharose, invert sugar, glucose, and so on, can be used appropriately.

When injectable products are formed, carriers, for example, water, alcohols, polyols, glycerols, vegetable oils, and so on, can be used appropriately.

When suppositories are formed, carriers, for example, vegetable oil, wax, oils, gels or liquid polyols and so on, can be used appropriately. Furthermore, these preparations can be used in combination with antiseptics, solvents, stabilizers, humectators, emulsifiers, edulcorants, bases for changing osmotic pressure, buffers, epiboly and antioxidants, and moreover, a therapeutically noteworthy compound.

The administration route of the above preparation is not deemed to be limited thereto, but may be adequately varied depending upon preparation forms, or age, sex, symptoms of patients, and so on.

The administration route, dosage and the number of administrations can be adequately varied depending upon the age, weight and symptoms of patients. In the case of oral administration, the dosage is usually 1 to 300 mg/kg (preferentially 3 to 30 mg/kg) per adult per day, and this dosage may be administered as a single daily dose or in multiple doses throughout the day.

When N-anisoyl-GABA or p-anisic acid was administered to rats orally, the acute toxicity (LD₅₀ values) of N-anisoyl-GABA was more than 5,000 mg/kg in both sexes, and that of p-anisic acid was 1,813 and 2,124 mg/kg in male and female animals, respectively. When both drugs were repeatedly administered orally to rats over a 4-week period, sub-acute toxicity, such as lethality and abnormality in hematological, hematobiochemical and toxipathological tests, was not observed up to 600 mg/kg.

The ACh release-enhancing effects by N-anisoyl-GABA and p-anisic acid and the improving effects of circadian rhythm disorder and sleep disorder by 1-p-anisoyl-2-pyrrolidinone are concretely explained below.

### EXAMPLE 1

Test of the enhancing effect of ACh release

Test animals: Male stroke-prone spontaneously hypertensive rats (SHRSP) at 13 weeks of age, which received 1% NaCl solution instead of water for 5 weeks prior to the experiment.

Test method: SHRSP were anesthetized and a guide cannula was implanted into the thalamus, dorsal hippocampus and prefrontal cortex. After recovery, a concentric microdialysis probe was inserted into the guide cannula and perfused with normal Ringer solution containing 10⁻⁵ M eserine (SIGMA, St. Louis, USA) at a constant flow rate of 2 μl/min under freely moving condition. N-anisoyl-GABA or p-anisic acid was dissolved in the Ringer solution at final concentrations of 10⁻⁷, 10⁻⁶ and 10⁻⁵ M, and each drug was perfused for 20 minutes through the same probe. The dialysates for every 20 minutes were collected and injected into the high-pressure liquid chromatography system to quantify extracellular ACh levels. ACh release was expressed as percent change over the average of three consecutive stable samples collected before drug perfusion.

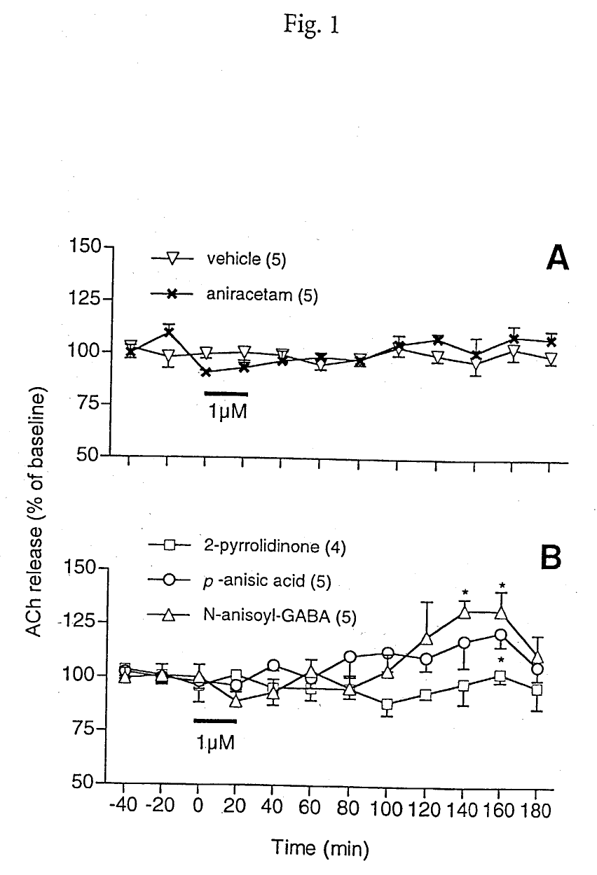

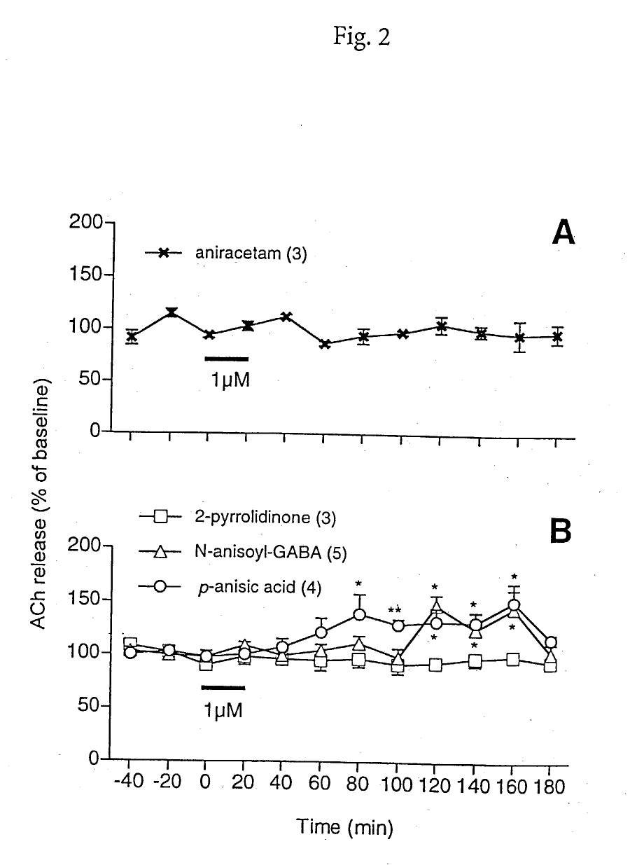

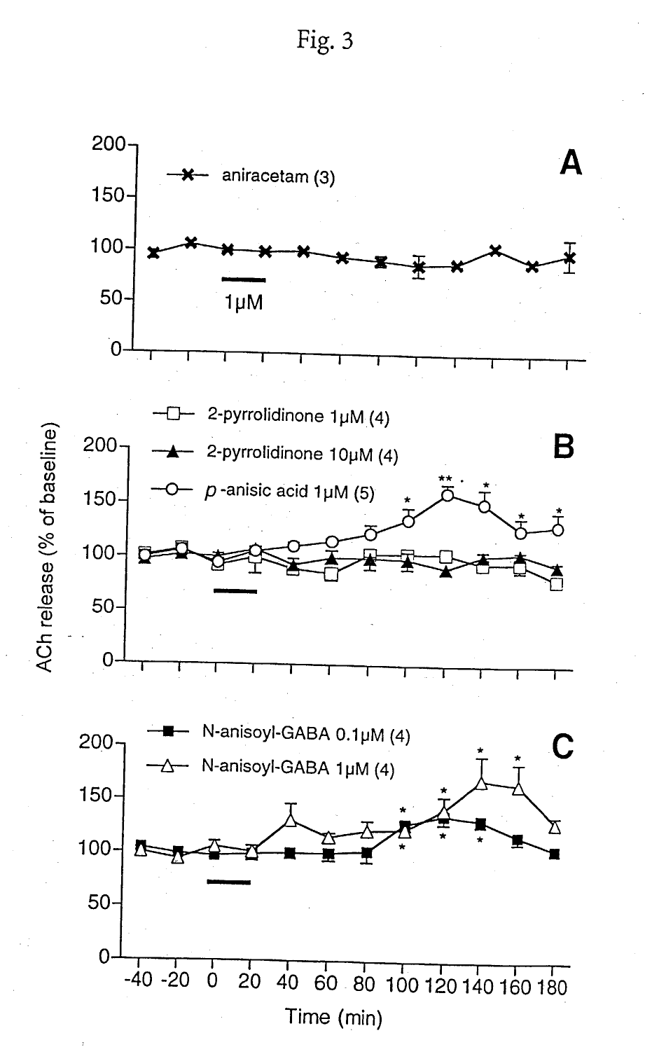

Test results: N-anisoyl-GABA enhanced ACh release by 32%, 48% and 70%, and p-anisic acid increased it by 22%, 51% and 61% in the thalamus (FIG. 1), dorsal hippocampus (FIG. 2) and prefrontal cortex (FIG. 3), respectively. On the contrary, the effect of 1-p-anisoyl-2-pyrrolidinone was not observed in any cerebral regions. Hence, N-anisoyl-GABA and p-anisic acid were assumed to be active substances contributing to the activation of central cholinergic neurons (enhancement of ACh release), as the major metabolites of 1-p-anisoyl-2-pyrrolidinone.

### EXAMPLE 2

Test of the ameliorating effect of impaired circadian rhythm

Test animals: Male Wistar rats at 9 weeks of age (young group) and around 30 months of age (aged group).

Test method: Animals were individually housed and had free access to food and water. After fasting for 24 hours (Day 0), feeding was restricted to only 1 hour/day from 13:30 for 6 consecutive days. Food was again withheld on Day 7, and food-motivated circadian anticipatory activity was investigated. 1-p-Anisoyl-2-pyrrolidinone at 30 and 100 mg/kg or vehicle was orally administered immediately after the feeding time, once daily for 7 consecutive days. Spontaneous motor activity was measured in each home cage.

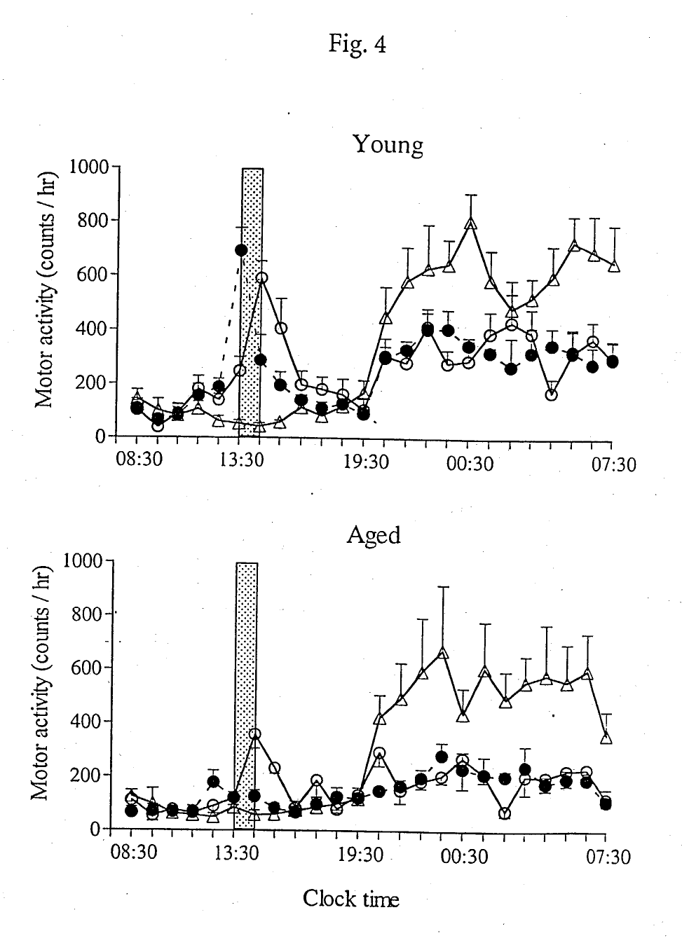

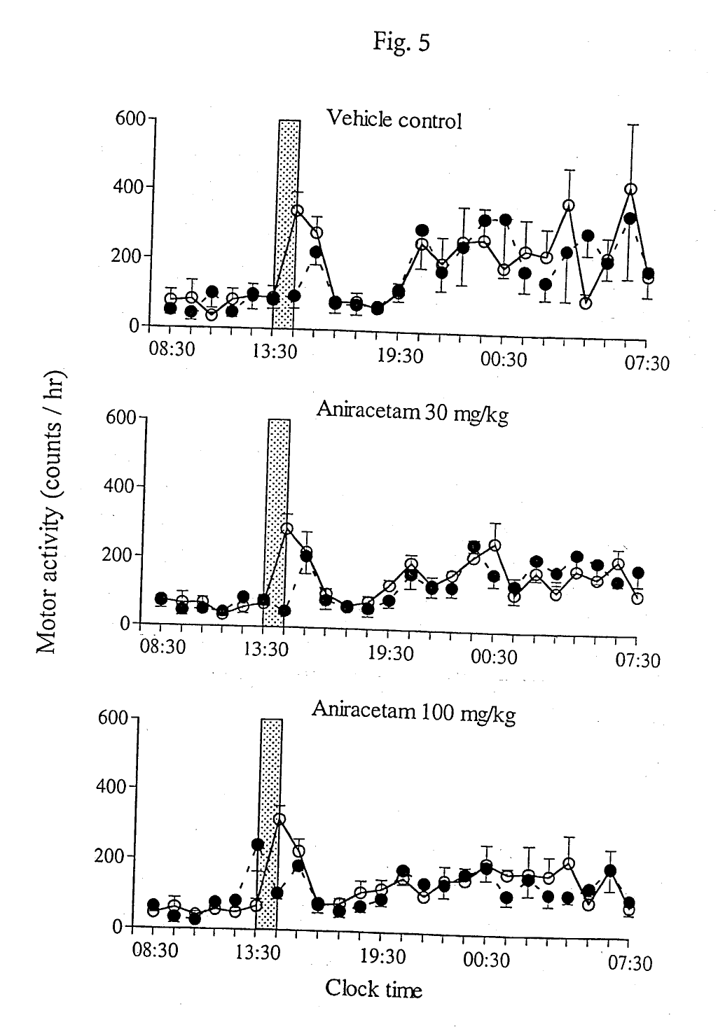

Test results: In aged rats, the food-motivated anticipatory activity on Day 7 was markedly attenuated as compared with that in young rats (FIG. 4), suggesting an abnormality in the regulation of circadian rhythm and deficiency of the time-keeping ability with aging. The repeated oral administration of 1-p-anisoyl-2-pyrrolidinone, but not vehicle, significantly ameliorated the impaired anticipatory activity in aged rats (FIG. 5).

### EXAMPLE 3

Test of improving effect of disturbed sleep pattern

Test animals: Male SHRSP at 13 weeks of age, which received 1% NaCl solution instead of water over 5 weeks, and Wistar Kyoto rats (WKY) at the same age.

Test method: Animals were anesthetized, and electrodes for electroencephalogram (EEG) and a sensor for brain temperature were implanted into the cerebral cortex and electrodes for electromyogram (EMG) were implanted into the back cervical muscle. All variables were continuously recorded for 7 days, and the behavioral states of rats were classified into wakefulness, REMS, NREMS by analyzing the amplitude and frequency of EEG and EMG waves. 1-p-Anisoyl-2-pyrrolidinone at 15 mg/kg or vehicle was administered orally twice daily (morning and evening) over 5 consecutive days.

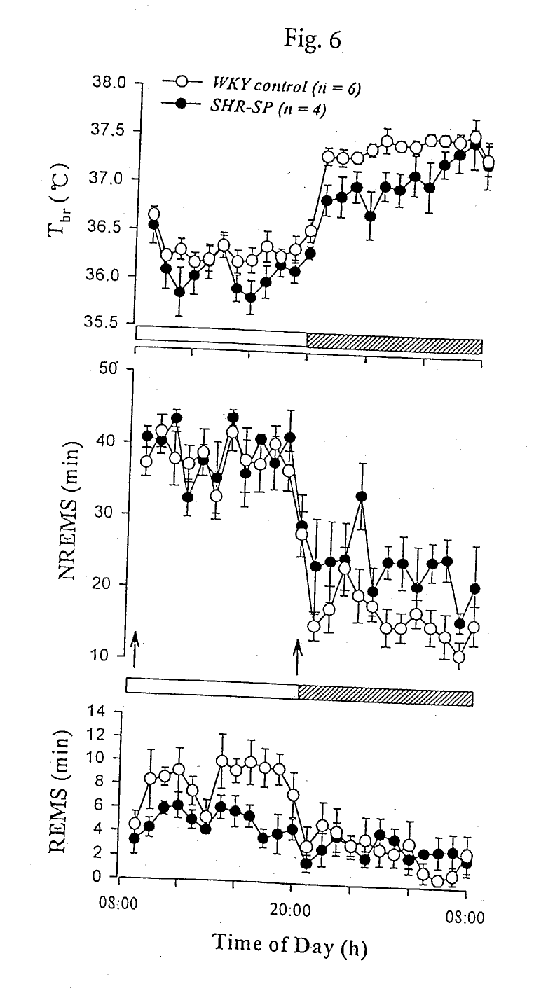

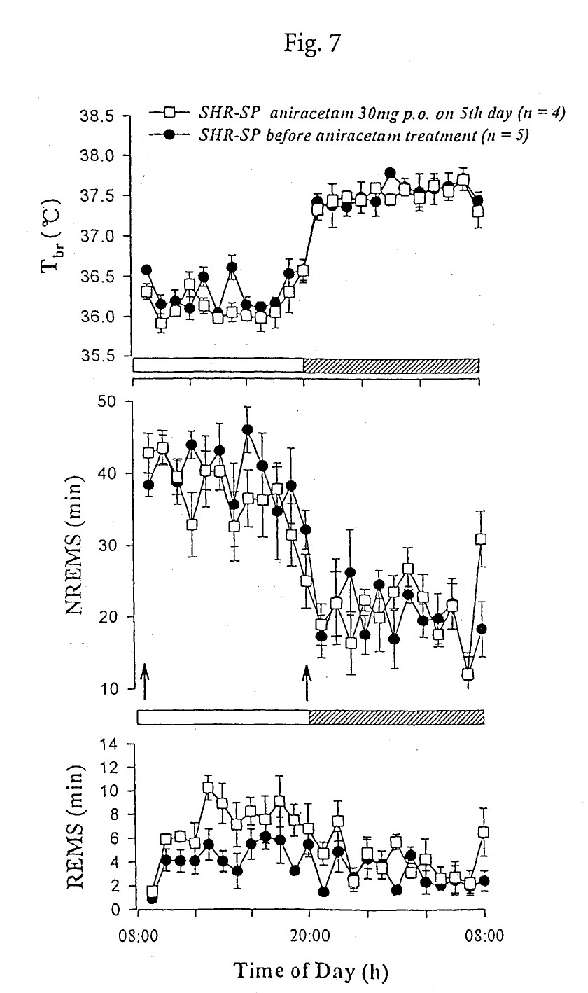

Test results: As compared with the control WKY, SHRSP showed a reduction in REMS during the light period (sleep period), and an increase in NREMS and decrease in brain temperature during the dark period (active period), indicating a disturbance of the sleep-waking rhythm (FIG. 6). The repeated oral administration of 1-p-anisoyl-2-pyrrolidinone, but not vehicle, ameliorated the reduced diurnal REMS in SHRSP (FIG. 7).

Based on the above it can be concluded that N-anisoyl-GABA or p-anisic acid is useful as a remedy for various neuropsychiatric symptoms, such as circadian rhythm disorder, sleep disorder, attention deficit disorder and problematic behaviors (delirium and nocturnal wandering), which are observed not only in cerebrovascular diseases (i.e., cerebral infarction and bleeding) but also in neuronal degeneration (i.e., Alzheimer's disease, Parkinson's disease and progressive supranuclear palsy) and hyperkinetic syndrome (attention-deficit hyperactivity disease). Furthermore, the combined effect of N-anisoyl-GABA and p-anisic acid can be sufficiently expected as well as each single effect.

### EXAMPLE 4

Preparation of pharmaceutical compositions

The pharmaceutically acceptable preparations as given below are suitable examples only and should not be considered to limit the pharmaceutical compositions of the present invention in any way.

Preparation of a tablet containing N-anisoyl-GABA

A tablet containing 100 mg of N-anisoyl-GABA is prepared by the following method using the following compositions (per tablet).

| Composition A: | | |
|---|---|---|
| N-anisoyl-GABA | 100 | mg |
| Lactose | 20 | mg |
| Kollidon CL (BASF) | 15 | mg |
| Corn starch | 30 | mg |
| Avicel PH 101 (AsahiChemical Co., Ltd.) | 50 | mg |
| Composition B: | | |
| Polyvinylpyrrolidinone K-90 | 5 | mg |
| Light anhydrous silicic acid | 18 | mg |
| Magnesium stearate | 2 | mg |
| Total | 240 | mg |

A mixture of composition A described above is kneaded in an 8% aqueous solution of polyvinylpyrrolidinone K-90. After drying at 60° C., composition B was mixed therewith. The mixture is tableted to a circular tablet weighing 240 mg and having a diameter of 8 mm.

Preparation of a tablet containing p-anisic acid

A tablet containing 100 mg of p-anisic acid is prepared by the following method using the following compositions (per tablet).

| Composition A: | | |
|---|---|---|
| p-Anisic acid | 100 | mg |
| Lactose | 20 | mg |
| Kollidon CL (BASF) | 15 | mg |
| Corn starch | 30 | mg |
| Avicel PH 101 (AsahiChemical Co., Ltd.) | 50 | mg |
| Composition B: | | |
| Polyvinylpyrrolidinone K-90 | 5 | mg |
| Light anhydrous silicic acid | 18 | mg |
| Magnesium stearate | 2 | mg |
| Total | 240 | mg |

A mixture of composition A described above is kneaded in an 8% aqueous solution of polyvinylpyrrolidinone K-90. After drying at 60° C., composition B was mixed therewith. The mixture is tableted to a circular tablet weighing 240 mg and having a diameter of 8 mm.

Preparation of a capsule containing N-anisoyl-GABA

A capsule containing 100 mg of N-anisoyl-GABA is prepared by the following method using the following compositions (per capsule).

| Composition A: | | |
|---|---|---|
| N-anisoyl-GABA | 100 | mg |
| Lactose | 20 | mg |
| Kollidon CL (BASF) | 2 | mg |
| Corn starch | 53 | mg |
| Composition B: | | |
| Polyvinylpyrrolidinone K-90 | 5 | mg |
| Avicel PH 101 (AsahiChemical Co., Ltd.) | 18 | mg |
| Magnesium stearate | 2 | mg |
| Total | 200 | mg |

A mixture of composition A described above is kneaded in an 8% aqueous solution of polyvinylpyrrolidinone K-90. After drying at 60° C., composition B was mixed therewith. The mixture is poured into a No. 3 gelatin capsule to obtain a capsule containing 200 mg.

1. A method of enhancing cerebral acetylcholine release in a mammal comprising administering to said mammal N-anisoyl-GABA or p-anisic acid and a therapeutically effective carrier in an amount effective for enhancing acetylcholine release.

2. The method of claim 1 comprising administering to said animal N-anisoyl-GABA.

3. The method of claim 1 comprising administering to said animal p-anisic acid.

4. The method of claim 1 wherein said N-anisoyl-GABA or p-anisic acid is administered to said mammal in a unit dosage from about 1 mg/kg per adult per day to about 300 mg/kg per adult per day.

5. A method of treating circadian rhythm disorders, sleep disorders, attention deficit disorders and problematic behaviors comprising administering to said mammal N-anisoyl-GABA or p-anisic acid and a therapeutically effective carrier in an amount effective for treating circadian rhythm disorders, sleep disorders, attention deficit disorders and problematic behaviors.

6. A pharmaceutical composition for enhancing cerebral acetylcholine release comprising a therapeutically effective amount of N-anisoyl-GABA or p-anisic acid.
